(19) United States
(12) Patent Application Publication
(10) Pub. No.: US 2025/0114413 A1
(43) Pub. Date: Apr. 10, 2025

(54) SYNBIOTIC SUPPLEMENT FORMULATION

(71) Applicant: MEDISYN-BIO INC., MASON, OH (US)

(72) Inventors: Saigiridhar TUMMALA, MASON, OH (US); Madhav DEVALARAJA, MASON, OH (US)

(21) Appl. No.: 18/483,005

(22) Filed: Oct. 9, 2023

(51) Int. Cl. A61K 35/747; A61K 31/35; A61K 31/702; A61K 31/716; A61K 31/733; A61K 35/745; A61K 36/45; A61K 36/48; A61P 1/00; C12R 1/25

(52) U.S. Cl. CPC A61K 35/747; A61K 31/35; A61K 31/702; A61K 31/716; A61K 31/733; A61K 35/745; A61K 36/45; A61K 36/48; A61P 1/00; C12R 2001/25

(57) ABSTRACT

The present disclosure relates to a formulation for supporting the growth or maintenance of bacteria in the gastrointestinal tract of an animal. Provided is the use of at least one human milk oligosaccharide in combination with pre-biotics and probiotic strains of bacteria for the manufacture of a food composition, pharmaceutical composition, food or dietary supplement, for the selective stimulation of beneficial bacteria in the gastrointestinal tract in context of a disease condition. Also provided is a synbiotic formulation comprising living beneficial bacteria formulated with at least one human milk oligosaccharide in combination with pre-biotics. In particular, synbiotic formulation is used for improving clinical biomarkers in diseases especially Crohn's disease.

### TECHNICAL FIELD

The present disclosure generally relates to a novel composition for supporting the growth or maintenance of bacteria in the gastrointestinal tract of an animal.

### BACKGROUND OF INVENTION

The role of microbiota in a variety of diseases is gradually emerging: chronic kidney disease (CKD), Diabetic Kidney Disease (DKD), Inflammatory or irritable bowel syndrome and Cancer show the presence of a dysregulation (so-called “dysbiosis”) of the gut microbiota composition and metabolism. Microbial metabolism represents the molecular link by which microbiota interacts with human physiology and diseases. This metabolism, mainly divided in saccharolytic or proteolytic, is generally believed to foster health when the balance is shifted towards the first one because of the different actions of the metabolic products derived from the two catabolic pathways. The saccharolytic one, in fact, mainly leads to the release of short-chain fatty acids (SCFA), known for their immune-modulating, anti-inflammatory and in general beneficial activities.

Dietary supplement or medical food formulas are commonly used today to provide a supplemental nutrition or modulation of biomarkers in certain conditions resulting from organ or physiological insufficiency. These formulas typically contain nutritional ingredients for certain function, and are commercially available as powders, ready-to-feed liquids, and liquid concentrates. Many supplement formulas provide a quality alternative as not all nutritional needs are met from regular diets.

In certain conditions, such as chronic kidney disease, chemotherapy, IBD, a dysbiotic gut is a common coexisting parameter. Ingredients of both pre and probiotics in a synbiotic formula that promote beneficial gut bacterial growth, metabolism, or both, improves one or more of clinical biomarkers in conditions of CKD, Chemotherapy and or IBD in human subjects.

Probiotics and their benefits for animal health are well known to skilled artisans. Probiotics are live microorganisms that have a beneficial effect in the prevention and treatment of specific medical conditions when ingested. Probiotics are believed to exert biological effects through a phenomenon known as colonization resistance. Probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Probiotics are known to enhance intestinal function, stimulate the immune system, reduce inflammation, and diminish the population of harmful microorganisms in the gastrointestinal tract.

Prebiotics are compounds in food that induce the growth or activity of beneficial microorganisms.

Mixtures of probiotics and prebiotics that beneficially affect the host by improving the survival and implantation of live microbial dietary supplements in the gastrointestinal tract, by selectively stimulating the growth and/or by activating the metabolism of one or a limited number of health-promoting bacteria, thus improving host welfare.

There is now a need for new compositions containing probiotics, particularly in the form of dietary supplements, that have enhanced therapeutic effects compared to previous probiotics alone, particularly in at least one of the conditions namely but not limited to Crohn's disease, chronic kidney disease (CKD), diabetic kidney disease (DKD), dysbiosis and or gut injury from chemotherapy, and inflammatory or irritable gut conditions of human subjects.

### BRIEF SUMMARY

In one or more embodiments, the present invention provides for dietary supplements and particularly to dietary supplements containing probiotics and prebiotics.

Some embodiments of the present disclosure include a composition comprising ingredients for a synbiotic formulation as medical food or dietary supplements that include at least one human milk oligosaccharide in combination with at least one pre-biotic and at least one probiotic strain of bacteria.

The present disclosure relates to a composition, which in some embodiments is a dietary supplement that may be useful for promoting beneficial gut bacterial growth, metabolism, or both, and that improves one or more of clinical biomarkers in conditions of CKD, Chemotherapy and or IBD in human subjects.

In one or more embodiments, the dietary supplement or medical food formula comprises at least one human milk oligosaccharide selected from 2′-fucosyllactose (2′-FL) and lacto-N-neotetraose (LNnT).

In one or more embodiments, the dietary supplement or medical food formula comprises at least one human milk oligosaccharide and a prebiotic selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g., guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. In some embodiments, the prebiotic is inulin. In another embodiment, the prebiotic is oligofructose-enriched inulin.

In one or more embodiments, the dietary supplement or medical food formula comprises at least one human milk oligosaccharide and a soluble fiber. In some embodiments, the soluble fiber is obtained from oats, barley, rye, wheat, seaweed, or mushroom. In some embodiments, the soluble fiber comprises beta-glucans, such as beta-D-glucose polysaccharides. β-Glucans (beta-glucans) comprise a group of β-D-glucose polysaccharides naturally occurring in the cell walls of bacteria, fungi, yeasts, algae, lichens, and plants, such as oats and barley.

In one or more embodiments, the dietary supplement or medical food formula comprises at least one prebiotic selected from oligofructose-enriched inulin (OFI) and beta-glucans (BG).

In one or more embodiments, the dietary supplement or medical food formula comprises the prebiotics oligofructose-enriched inulin (OFI) and beta-glucans (BG).

In one or more embodiments, the dietary supplement or medical food formula comprises the prebiotics oligofructose-enriched inulin (OFI) and beta-glucans (BG) and at least one additional prebiotic selected from astaxanthin, glucosamine, and an omega-3 fatty acid.

In one embodiment of the foregoing aspect, the prebiotic further comprises a monomer or polymer selected from the group consisting of arabinoxylan, xylose, soluble fiber dextran, soluble corn fiber, polydextrose, lactose, N-acetyl-lactosamine, glucose, and combinations thereof. In one embodiment of the foregoing aspect, the prebiotic comprises a monomer or polymer selected from the group consisting of galactose, fructose, rhamnose, mannose, uronic acids, 3′-fucosyllactose, 3′sialylactose, 6′-sialyllactose, lacto-N-neotetraose, 2′-2′-fucosyllactose, and combinations thereof. In one embodiment of the foregoing aspect, the prebiotic comprises a monosaccharide selected from the group consisting of arabinose, fructose, fucose, lactose, galactose, glucose, mannose, D-xylose, xylitol, ribose, and combinations thereof. In one embodiment of the foregoing aspect, the prebiotic comprises a disaccharide selected from the group consisting of xylobiose, sucrose, maltose, lactose, lactulose, trehalose, cellobiose, and combinations thereof. In one embodiment of the foregoing aspect, the prebiotic comprises a polysaccharide, wherein the polysaccharide is xylooligosaccharide.

In one embodiment of the foregoing aspect, the prebiotic comprises a sugar selected from the group consisting of arabinose, fructose, fucose, lactose, galactose, glucose, mannose, D-xylose, xylitol, ribose, xylobiose, sucrose, maltose, lactose, lactulose, trehalose, cellobiose, xylooligosaccharide, and combinations thereof.

In one or more embodiments, the present disclosure is directed to dietary supplement or medical food formula that include a combination of at least one human milk oligosaccharide selected from 2′-fucosyllactose (2′-FL) and lacto-N-neotetraose (LNnT), in combination with oligofructose-enriched inulin (OFI), or beta-glucans (BG) or probiotic strains of Bifidobacterium and Lactobacilli strains, where the OFI and BG are present at 3-14 gm and 1-5 gm respectively.

Also provided are methods of use of the supplement formulation for improving biomarkers in at least one of the conditions namely Chronic Kidney Disease (CKD), Diabetic Kidney Disease (DKD), dysbiosis and or Gut injury from Chemotherapy, and inflammatory or irritable gut conditions of human subjects.

In one or more embodiments, the dietary supplement or medical food formulas and methods of the present disclosure offer an alternative therapeutic or nutritional intervention option that can contribute to one or more of improved clinical biomarkers in at least one of the conditions namely Chronic Kidney Disease (CKD), Diabetic Kidney Disease (DKD), dysbiosis and or Gut injury from Chemotherapy, and inflammatory or irritable gut conditions in children, youth or adult humans.

In one or more embodiments, the present invention provides for the administration of the compositions described further comprising vitamins, minerals including necessary electrolytes, super anti-oxidants, proteins, amino acids, complex carbohydrates, fiber, lipids and bio-flavonoids with superior absorption, utilization and retention.

In one or more embodiments, the present invention provides for the methods provided herein include administering the compositions as a health bar, grain or drink.

In an embodiment, a synbiotic formulation is used as dietary supplement or medical food that include at least one human milk oligosaccharide (HMO) selected from 2′-fucosyllactose (2′-FL) and/or lacto-N-neotetraose (LNnT), in combination with astaxanthin and Probiotic strains of Bifidobacterium and Lactobacilli strains (20 billion CFU each), where the HMO and astaxanthin are present at 0.5-10 g and 0.01-2 g respectively.

In an embodiment, the synbiotic formulation comprising between about 1 g/mL and about 10 g/mL of at least one human milk oligosaccharide.

In an embodiment, the synbiotic formulation comprising between about 1 g/mL to about 2 g/mL of at least one human milk oligosaccharide.

In an embodiment, the synbiotic formulation comprises HMO consists 2′FL.

In an embodiment, the synbiotic formulation further comprises inulin and betaglucan.

In an embodiment, inulin is present in the synbiotic formulation as combination of long and short chains, in oligofructose enriched form; where the beta glucan is originated from oat or barley or as a mixture thereof.

In an embodiment, inulin is present between about 3 g/mL to about 7 g/mL and betaglucan is present between about 0.4 g/mL to about 1 g/mL.

In an embodiment, the synbiotic formulation further comprises SunCran®.

In an embodiment, the suncran is present between 1 g/mL to about 4 g/mL.

In an embodiment, the synbiotic formulation is used to improve clinical biomarkers for Crohn's disease.

In an embodiment, synbiotic formulation increase iron content in the body.

In an embodiment, synbiotic formulation increase iron absorption in the body and is used to treat anemia.

In an embodiment, synbiotic formulation has an effect on colon, serum biomarkers and reduce inflammation, inflammation biomarkers such as CRP (C-Reactive Protein), calprotectin, anemia.

In an embodiment, synbiotic formulation reduce fecal calprotectin.

In an embodiment, in the synbiotic formulation 2FL (HMO), Astaxanthin, Probiotics-Bifidobacter bifidum, Bifidobacter longum, Lactobacillus plantarum, Lactobacillus rhamnosus are essential ingredients.

In an embodiment, method of treatment for Crohn's disease comprises administering 2 sachets (each sachet is about 10 g) dissolved in water, for at least 8 weeks. Further, based on the severity, dosage can be vary e.g. for healthy person one sachet is may be enough and for a sever-ill person, two dosages are needed.

In an embodiment, method of treatment comprising administration of the synbiotic formulation is for 7 years and above.

In an embodiment, the synbiotic formulation is supplemented with collagen peptides for treating knee pain or arthritis.

In another embodiment, the synbiotic formulation comprises citrus flavanoids.

In an embodiment, the synbiotic formulation is supplemented with citronella, and betain i.e. beet root extract for heart and cholesterol and blood pressure control

In an embodiment, the synbiotic formulation comprises citronel for glucose control, rheomin for improving kidney functions.

In another embodiment, the synbiotic formulation comprises melatonin for gut health and sleep medication; niacin i.e. vitamin B3 for brain development and for improving memory and cognitive functions; glutanin and xanthin for improving eye health for vision and prebiotics for pregnant women.

In an embodiment, the synbiotic formulation comprises fillers and binders.

In an embodiment, the synbiotic formulation comprises the pharmaceutical excipients that can be used are one or more fillers, one or more binders or diluents, one or more lubricants, one or more disintegrants, and one or more lubricants, one or more film coatings, one or more plasticizer or 1 or more types of pigments or 1 or more flavor.

The pharmaceutical composition (tablet) of the present invention usually contains a binder.

In an embodiment, the synbiotic formulation is in the form of powder, solution, paste, chewable gummy, decoction, tablet, bar.

In an embodiment, the synbiotic formulation is prepared in water or organic solvent or mixture of both.

In an embodiment, probiotic strains are freeze dried (lyophilized), attenuated, refrigerated, cryopreserved before mixing it in the synbiotic formulation.

In an embodiment, probiotic strain comprises at least 1 billion microorganisms of each strain.

In an embodiment, synbiotic formulation comprising probiotic strains is stored at room temperature.

In an embodiment, the life of synbiotic formulation is at least one year, when stored at dried place at room temperature.

In an embodiment, the life of synbiotic formulation is at least two years, when stored at dried place at room temperature.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The figures are furnished with the application to understand the invention sought to be patented. It shall not be construed as the only way to perform the invention sought to be patented.

FIG. 1 shows colonoscopy results, after 12 weeks of daily administration of synbiotic formulation, demonstrates complete normalization of ascending colon.

FIG. 2 shows complete reduction of the classic disease marker-fecal calprotectin after 12 weeks of daily administration of synbiotic formulation.

### DETAILED DESCRIPTION

### Definitions

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” or “about” can, in some embodiments, mean within plus or minus ten percent of the stated value. In other embodiments, “approximately” or “about” can mean within plus or minus five percent of the stated value. In further embodiments, “approximately” or “about” can mean within plus or minus three percent of the stated value. In yet other embodiments, “approximately” or “about” can mean within plus or minus one percent of the stated value.

Unless otherwise mentioned, all numeric values mentioned in the specification to be construed with approximate variations as would be understood by a person skilled in the art.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The following terms and phrases, unless otherwise indicated, shall be understood to have the following meanings.

“Administering” a composition can be effected or performed using any of the various methods and delivery systems known to those skilled in the art. In an embodiment, administration includes an oral administration.

The term “subject” means a human or other animal, including avian, bovine, canine, equine, feline, hicrine, murine, ovine, and porcine animals, that can benefit from the administration of probiotics and prebiotics.

The term “bioavailability” includes, generally, the degree to which a drug or other substance becomes available to a subject following ingestion, administration, or exposure. In one embodiment, for example, the bioavailability of the quercetin compounds may be the bioavailability to a particular target tissue. For example, in an embodiment, the particular target tissue may require traversal of the stomach or the small intestines, therefore the bioavailability data may be obtained from this particular target tissue.

The term “biomarkers” or “clinical biomarkers” as used herein, refer to a measurable entity of the present invention that has been determined to be predictive of the effects of the prebiotic and probiotic bacteria therapy described herein, either alone or in combination with at least one other therapy on a target disease or disorder (e.g., one of inflammatory and/or immune diseases or disorders, such as cancers). Biomarkers can include, without limitation, increased glomerular filtration rate, reduced Urinary albumin, reduced Trimethlyamine N-oxide (TMAO), reduced interlukin-6 (IL-6), reduced C-reactive protein (CRP), reduced tumor necrosis factor alpha (TNF-alpha, improved stool consistency, improved regularity of bowel movements, reduce diarrhea, improved nutrient absorption from gut, increased villi length in gut and combinations thereof.

The term “chronic kidney disease” or “CKD”, as used herein, refers generally to individuals where kidneys are damaged and can't filter blood the way they should. The main risk factors for developing kidney disease include but are not limited to diabetes, high blood pressure, heart disease, and a family history of kidney failure.

As used herein, chemical compounds or simply “compounds” may be identified either by their chemical structure, chemical name, or common name. In the event that the chemical structure, chemical name, or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers, or diastereomers. Accordingly, the chemical structures depicted herein encompass all possible enantiomers and stereoisomers of the illustrated or identified compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, 2H, 3H, 13C, 14C, 15N, 180, 170 etc. Compounds may exist in unsolvated forms as well as solvated forms, including hydrated forms and as N-oxides. In general, compounds may be hydrated, solvated, or N-oxides. Certain compounds may exist in multiple crystalline or amorphous forms. Also contemplated are congeners, analogs, hydrolysis products, metabolites, and precursor or prodrugs of the compound. In general, all physical forms are equivalent for the uses contemplated herein and are intended to be within the scope of the present disclosure.

Concentrations, amounts, and other numerical data may be expressed or presented herein in a range format. It is to be understood that such a range format is used merely for convenience and brevity and thus should be interpreted flexibly to include not only the numerical values explicitly recited as the limits or endpoints of the range, but also to include all the individual numerical values and/or sub-ranges encompassed within that range as if each numerical value (including fractions) and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 2.6, 3, 3.8, and 4 and sub-ranges such as from 1-3, from 2-4, and from 3-5, etc., as well as 1, 2, 3, 4, and 5, individually.

As used herein a “concentrate” refers to an extract of a source that contains at least the same amount of active fractions, compounds, or other constituents, in a smaller volume than in the source itself. In one example, a “concentrate” may be a dried powder derived from a component that does not include the use of any solvents during the concentration process.

The term “dietary supplement” means a product that is intended to be ingested in addition to the normal diet of an animal.

The term “dosage unit” is understood to mean a unitary, i.e. a single dose which is capable of being administered to a subject or patient, and that may be readily handled and packed, remaining as a physically and chemically stable unit dose comprising either the active ingredient as such or a mixture of it with solid or liquid pharmaceutical vehicle materials.

An “effective amount,” “therapeutic amount,” “therapeutic effective amount,” or “effective dose” is an amount or dose sufficient to elicit a desired pharmacological or therapeutic effect in a mammalian subject. Therapeutic effectiveness may further be demonstrated by a decrease in the symptoms of the conditions being treated. An “effective amount” of a composition means an amount of the composition sufficient to cause the desired result in a given subject. The effective amount will vary from subject to subject and depending on the condition to be treated, the agent delivered, and the route of delivery. A person of ordinary skill in the art can perform routine titration experiments to determine such an amount. Depending upon the agent delivered, the effective amount of a composition can be delivered continuously, such as by a suppository, or at periodic intervals (for example, on one or more separate occasions). Desired time intervals of multiple amounts of a particular agent can be determined without undue experimentation by one skilled in the art.

The term “food” includes all edible compositions regardless of form and thus includes gels, gel packs, liquids, syrups, and/or solids. A “food” or “food product” or “food composition” means a product or composition that is intended for ingestion by an animal, including a human, and provides nutrition to the animal.

A “fucosylated oligosaccharide” is an oligosaccharide having a fucose residue. It has a neutral nature. Some examples are 2′-FL (2′-fucosyllactose or 2-fucosyllactose or 2FL or 2-FL), 3-FL (3-fucosyllactose), difucosyllactose, lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

The expressions “galacto-oligosaccharide”, “galactooligosaccharide” and “GOS” can be used interchangeably. They refer to an oligosaccharide comprising two or more galactose molecules which has no charge and no N-acetyl residue (i.e. they are neutral oligosaccharide). In a particular embodiment, said two or more galactose molecules are linked by a beta-1,2, beta-1,3, beta-1,4 or beta-1,6 linkage. In another embodiment, “galacto-oligosaccharide” and “GOS” also include oligosaccharides comprising one galactose molecule and one glucose molecule (i.e. disaccharides) which are linked by a beta-1,2, beta-1,3 or beta-1,6 linkage.

The term “gut injury from chemotherapy” as used herein, refers to individuals where chemotherapy leads to gastrointestinal (GI) mucositis with nonspecific symptoms such as nausea, vomiting, abdominal pain and diarrhea that are reported in almost all chemotherapy recipients. Histological GI mucositis lesions typically are represented by villus atrophy and loss of enterocytes, resulting in epithelium impairment and barrier dysfunction.

The term “HMO” or “HMOs” refers to human milk oligosaccharide(s). These carbohydrates are resistant to enzymatic hydrolysis by digestive enzymes (e.g pancreatic and/or brush border), indicating that they may display functions not directly related to their caloric value. It has especially been illustrated that they play a vital role in the early development of infants and young children, such as the maturation of the immune system. Many different kinds of HMOs are found in the human milk. The HMOs can be acidic (e.g. charged sialic acid containing oligosaccharide) or neutral (e.g. fucosylated oligosaccharide).

The term “inflammatory bowel disease,” “irritable bowel disease” or “IBD”, as used herein refers to, involves chronic inflammation of part of the digestive tract. Examples of this type of disease include Crohn's disease and ulcerative colitis. The condition is a chronic disorder that interferes with the normal functions of the colon and is characterized by symptoms such as abdominal pain, cramping, bloating, constipation, and diarrhea.

A “medical food” as used herein refers to a specially formulated product and intended for the dietary management of diseases or medical conditions (e.g., to prevent or treat undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution. A food product, medical food or nutritional composition can be in any oral nutritional form, e.g. as a health drink, as a ready-made drink, optionally as a soft drink, including juices, milk-shake, yogurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), and dairy products.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) or any combination thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The term “oligosaccharide” means a carbohydrate having a degree of polymerization (DP) ranging from 2 to 20 inclusive but not including lactose. In some embodiments of the invention, carbohydrate has DP ranging from 3 to 20. The expressions “at least one N-acetylated oligosaccharide, one galacto-oligosaccharide and one sialylated oligosaccharide” and “at least one N-acetylated oligosaccharide, at least one galacto-oligosaccharide and at least one sialylated oligosaccharide” can be used interchangeably. The expressions “oligosaccharide(s) mixture” or “mixture of oligosaccharide(s)” can be used interchangeably. The oligosaccharide(s) mixture according to one aspect of the invention comprises at least one N-acetylated oligosaccharide, at least one galacto-oligosaccharide and at least one sialylated oligosaccharide. The mixture may be made of one or several oligosaccharides of these different types, i.e. one or several N-acetylated oligosaccharide(s), one or several galacto-oligosaccharide(s) and one or several sialylated oligosaccharide(s). In some embodiments the oligosaccharides of the oligosaccharide mixture are bovine's milk oligosaccharides (or BMOs).

The term “percent by weight” of a oligosaccharide (HMO), beta glucan or inulin, means the weight of such ingredients as measured by high-performance liquid chromatography (HPLC), sometimes referred to as high-pressure liquid chromatography. While it is also possible to measure the percent by weight of HMO, beta glucan or Inulin by using UV absorption techniques, such techniques detect ancillary materials and therefore report a higher and inaccurate percentage by weight than the HPLC method of measurement. Therefore, to provide the most accurate disclosure, all measurements and reporting of percentages by weight are done using HPLC.

As used herein, the term “prevention” refers to any activity that reduces the burden of the individual later expressing disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

The term “prebiotic” in certain instances may refer to food ingredients or bacterial producing ingredients that are not readily digestible by endogenous host enzymes and confer beneficial effects on an organism that consumes them by selectively stimulating the growth and/or activity of a limited range of beneficial microorganisms, such as those that are associated with the intestinal tract. Benefits derived from prebiotics facilitated establishment of probiotic microorganisms within the digestive tract include reduction of pathogen load, improved microbial fermentation patterns, improved nutrient absorption, improved immune function, improved intestinal hormonal signaling and metabolic regulation, aided digestion, increasing health and fitness. Probiotics are sometimes combined with prebiotics (where the combination may be referred to as ““synbiotic”) which include one or more non-digestible dietary supplements, which modify the balance of the intestinal micro flora, stimulating the growth and/or activity of beneficial microorganisms and suppressing potentially deleterious microorganisms. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS). Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or oral dietary supplement).

The term “probiotic” means any microorganism that exerts a beneficial effect on the host animal such as increased health or resistance to disease. Probiotics can exhibit one or more of the following non-limiting characteristics: non-pathogenic or non-toxic to the host; are present as viable cells, preferably in large numbers; capable of survival, metabolism, and persistence in the gut environment (e.g., resistance to low pH and gastrointestinal acids and secretions); adherence to epithelial cells, particularly the epithelial cells of the gastrointestinal tract; microbicidal or microbiostatic activity or effect toward pathogenic bacteria; anticarcinogenic activity; immune modulation activity, particularly immune enhancement; modulatory activity toward the endogenous flora; enhanced urogenital tract health; antiseptic activity in or around wounds and enhanced would healing; reduction in diarrhea; reduction in allergic reactions; reduction in neonatal necrotizing enterocolitis; reduction in inflammatory bowel disease; and reduction in intestinal permeability. Examples of probiotics include but are not limited to Bifidobacterium spp., Lactobacillus spp., Streptococcus thermophilia, Bacillus coagulans, Bacillus laterosporus, Pediococcus acidilactici, Saccharomyces boulardii. Examples of probiotic Lactobacillus spp. include but are not limited to Lactobacillus plantarum, Lactobacillus acidophilus, Lactobacillus crispatus, Lactobacillus casei, and Lactobacillus rhamnosus. Bifidobacterium spp. are commensal to the gut and are commonly used in probiotic supplements. Examples of probiotic Bifidobacterium spp. include but are not limited to Bifidobacterium longum, Bifidobacterium lactus, Bifidobacterium infantis, Bifidobacterium breve.

A “sialylated oligosaccharide” is a charged sialic acid containing oligosaccharide, i.e. an oligosaccharide having a sialic acid residue. It has an acidic nature. Some examples are 3-SL (3′ sialyllactose) and 6-SL (6′ sialyllactose).

The term “statistically significant” or “significantly” refers to statistical evidence that there is a difference. It is defined as the probability of making a decision to reject the null hypothesis when the null hypothesis is actually true. The decision is often made using the p-value.

The term “synbiotics” as used herein refers to combinations of prebiotics and probiotics that are believed to have a synergistic effect by inhibiting the growth of pathogenic bacteria and enhancing the growth of beneficial organisms.

As used herein, the term “treatment” refers to an intervention made in response to a disease, disorder or physiological condition manifested by a subject, particularly a subject suffering from one or more of a disorder, disease or disease state. In some embodiments, the disorder, disease or disease state is a heart disease, diabetes, hypertension, allergic reactions, asthma, arthritis, cancer, prostate diseases, chronic kidney disease, dry eye disease or syndrome and oxidative stress. In some embodiments, the treatment refers to the improvement of health or performance, including the improvement of athletic performance, improved bone health, mental health, strengthened immune response, prevention of fatigue, reduction in recovery time following exercise, and for boosting energy. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to therapeutic, prophylactic and/or supportive and adjunct therapy measures. Those in need of treatment include those already affected by a disease or disorder or undesired physiological condition as well as those in which the disease or disorder or undesired physiological condition is to be prevented. For example, in some embodiments, treatments reduce, alleviate, or eradicate the symptom(s) of the disease(s). In some embodiments, treatment can refer to enhancement of secondary effects of the disease, including enhancement or improvement of athletic performance.

The term “viable” means alive and capable of reproduction or colonization.

The term “Crohn's disease” is a type of inflammatory bowel disease (IBD). It causes inflammation of digestive tract, which can lead to abdominal pain, severe diarrhea, fatigue, weight loss, anemia and malnutrition. In an embodiment, increase in fecal calprotectin is a clinical biomarker of this disease. In an embodiment, in Crohn's disease there are multiple granulomas in terminal ileum and entire colon.

Some embodiments of the present disclosure include a composition comprising ingredients for a synbiotic formulation as medical food or dietary supplements that include at least one human milk oligosaccharide in combination with pre-biotics and probiotic strains of bacteria.

The present disclosure relates to a composition, which in some embodiments is a dietary supplement and/or medical food that may be useful for promoting beneficial gut bacterial growth, metabolism, or both, and that improves one or more of clinical biomarkers in conditions of CKD, Chemotherapy and or IBD in human subjects.

Some embodiments of the present disclosure promote growth of beneficial gut flora and reduce the toxic bacterial metabolites evidenced in certain disease conditions. This embodiment also promotes improvement of gut health.

Some embodiments of the present disclosure can be consumed daily for its preventive health benefits, as needed for therapy or as an adjunct to ongoing clinical therapy.

### Formulation

In one or more embodiments, the composition comprises at least the active ingredients at least one human milk oligosaccharide in combination with at least one pre-biotic and at least one probiotic strain of bacteria, in varying concentrations.

In one or more embodiments, the dietary supplement or medical food formula comprises at least one human milk oligosaccharide selected from 2′-fucosyllactose (2′-FL) and lacto-N-neotetraose (LNnT).

In one or more embodiments, the dietary supplement or medical food formula comprises at least one human milk oligosaccharide and a prebiotic selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g., guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. In some embodiments, the prebiotic is inulin. In another embodiment, the prebiotic is oligofructose-enriched inulin.

In one or more embodiments, the dietary supplement or medical food formula comprises at least one human milk oligosaccharide and a prebiotic soluble and or insoluble fiber.

Soluble fibers largely consist of polysaccharide carbohydrates that can form a gel during digestion, while the insoluble fibers add bulk to the stool formation in digestive tract.

Oats/oat bran, dried beans and peas, nuts, barley, flaxseed, fruits such as oranges and apples, vegetables such as carrots and psyllium husks are good sources of soluble fiber.

The soluble fibers from oats, barley, rye, wheat, some types of seaweed, and various species of mushrooms contain larger amounts of beta-glucans, that consist of various molecular weight polymers of beta-D-glucose polysaccharides. Daily consumption of beta-glucans has potential positive health effects, particularly in reducing the risk of heart disease.

Soluble fibers are generally separated from the above-mentioned sources by mechanical and/or wet processing techniques that frequently include enzymatic digestion of longer chain polysaccharide carbohydrate molecules present in the source material. The enzymatic digestion serves to assist in the separation and concentration of the soluble fiber fractions including beta-glucans.

The amount of soluble fibers can vary in the dietary supplement or medical food formula. In some embodiments, the dietary supplement or medical food formula includes from about 0.05% (w/w) to about 3% (w/w) soluble fiber. In some embodiments, the dietary supplement or medical food formula includes from about 0.1% (w/w) to about 2.5% (w/w) soluble fiber. In some embodiments, the dietary supplement or medical food formula includes from about 0.2% (w/w) to about 1.5% (w/w) soluble fiber. In some embodiments, the dietary supplement or medical food formula includes from about 0.3% (w/w) to about 1.2% (w/w) soluble fiber. In some embodiments, the dietary supplement or medical food formula includes from about 0.4% (w/w) to about 1% (w/w) soluble fiber. In some embodiments, the dietary supplement or medical food formula includes from about 0.5% (w/w) to about 0.9% (w/w) soluble fiber. In some embodiments, the dietary supplement or medical food formula includes from about 0.6% (w/w) to about 0.8% (w/w) soluble fiber. In some embodiments, the dietary supplement or medical food formula includes at least about 0.05%, 0.1%, 0.2%, 0.3%, 0.4%, 0.5%, 0.6%, 0.7%, 0.8% or 0.9% (w/w) soluble fiber.

In some embodiments, the dietary supplement or medical food formula includes no more than about 3%, 2.5%, 2%, 1.5%, 1.2%, 1.1%, 1%, 0.9%, 0.8%, 0.7%, 0.6%, 0.5%, 0.4%, 0.3%, 0.2% or 0.1% (w/w) soluble fiber.

Soluble fibers can consist of beta-glucan, maltodextrins and other carbohydrates, plant proteins, pectin, gums, inulin-type fructans and some hemicelluloses (e.g., arabinoxylan), among others.

Beta-glucan is a linear polysaccharide of glucose monomers with beta (1,4) and beta (1,3) linkages and found in the endosperm of cereal grains, primarily barley and oats. In some embodiment, the dietary supplement or medical food formula includes from about 0.01% (w/w) to about 2% (w/w) beta-glucan. In some embodiments, the dietary supplement or medical food formula includes from at least about 0.01%, 0.02%, 0.03%, 0.04%, 0.05%, 0.06%, 0.07%, 0.08%, 0.09%, 0.1%, 0.15%, 0.2%, 0.3%, 0.4%, or 0.5% (w/w) beta-glucan. In some embodiments, the dietary supplement or medical food formula includes no more than about 2%, 1%, 0.5%, 0.4%, 0.3%, 0.2%, 0.1%, 0.09%, 0.08%, 0.07%, 0.06%, 0.05%, 0.04%, 0.03%, or 0.02% (w/w) beta-glucan.

Maltodextrins and other carbohydrates and plant proteins also generally form part of commercially available soluble fibers and their content in the soluble fiber can be largely determined by the effectiveness of the separation and concentration processes used in the manufacture of the soluble fiber. In some embodiments, the soluble fiber used in the dietary supplement or medical food formula can contain up to 10% plant protein, and 55% maltodextrins and other carbohydrates. In some embodiments, the soluble fiber used in the dietary supplement or medical food formula can contain only 0.5% plant protein and less than 30% of maltodextrins and other carbohydrates.

In some embodiments, the composition includes from about 0.001% (w/w) to about 1% (w/w) plant proteins. In some embodiments, the dietary supplement or medical food formula includes from at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.15%, 0.2%, 0.3%, 0.4%, or 0.5% (w/w) plant proteins. In some embodiments, the dietary supplement or medical food formula includes no more than about 1%, 0.5%, 0.4%, 0.3%, 0.2%, 0.1%, 0.09%, 0.08%, 0.07%, 0.06%, 0.05%, 0.04%, 0.03%, 0.02%, 0.01%, or 0.005% (w/w) plant proteins.

Inulin is a polymer of fructose monomers. In some embodiments, the dietary supplement or medical food formula includes from about 0.001% (w/w) to about 1% (w/w) inulin. In some embodiments, the dietary supplement or medical food formula includes from at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.15%, 0.2%, 0.3%, 0.4%, or 0.5% (w/w) inulin. In some embodiments, the dietary supplement or medical food formula includes no more than about 1%, 0.5%, 0.4%, 0.3%, 0.2%, 0.1%, 0.09%, 0.08%, 0.07%, 0.06%, 0.05%, 0.04%, 0.03%, 0.02%, 0.01%, or 0.005% (w/w) inulin.

In some embodiments, the pharmaceutical composition may further comprise a prebiotic selected from the group consisting of: complex carbohydrates, complex sugars, resistant dextrins, resistant starch, amino acids, peptides, nutritional compounds, biotin, polydextrose, fructooligosaccharide (FOS), galactooligosaccharides (GOS), inulin, starch, lignin, psyllium, chitin, chitosan, gums (e.g., guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. In some embodiments, the prebiotic is inulin. In another embodiment, the prebiotic is oligofructose-enriched inulin.

In some embodiments, the soluble fiber is obtained from oats, barley, rye, wheat, seaweed, or mushroom. In some embodiments, the soluble fiber comprises beta-glucans, such as beta-D-glucose polysaccharides. β-Glucans (beta-glucans) comprise a group of β-D-glucose polysaccharides naturally occurring in the cell walls of bacteria, fungi, yeasts, algae, lichens, and plants, such as oats and barley.

In one or more embodiments, the dietary supplement or medical food formula comprises at least one prebiotic selected from oligofructose-enriched inulin (OFI) and beta-glucans (BG).

In one or more embodiments, the dietary supplement or medical food formula comprises the prebiotics oligofructose-enriched inulin (OFI) and beta-glucans (BG).

In one or more embodiments, the dietary supplement or medical food formula comprises the prebiotics oligofructose-enriched inulin (OFI) and beta-glucans (BG) and at least one additional functional ingredient selected from astaxanthin, glucosamine, and an omega-3 fatty acid.

In one embodiment of the foregoing aspect, the prebiotic further comprises a monomer or polymer selected from the group consisting of arabinoxylan, xylose, soluble fiber dextran, soluble corn fiber, polydextrose, lactose, N-acetyl-lactosamine, glucose, and combinations thereof. In one embodiment of the foregoing aspect, the prebiotic comprises a monomer or polymer selected from the group consisting of galactose, fructose, rhamnose, mannose, uronic acids, 3′-fucosyllactose,3′sialylactose, 6′-sialyllactose, lacto-N-neotetraose, 2′-2′-fucosyllactose, and combinations thereof. In one embodiment of the foregoing aspect, the prebiotic comprises a monosaccharide selected from the group consisting of arabinose, fructose, fucose, lactose, galactose, glucose, mannose, D-xylose, xylitol, ribose, and combinations thereof. In one embodiment of the foregoing aspect, the prebiotic comprises a disaccharide selected from the group consisting of xylobiose, sucrose, maltose, lactose, lactulose, trehalose, cellobiose, and combinations thereof. In one embodiment of the foregoing aspect, the prebiotic comprises a polysaccharide, wherein the polysaccharide is xylooligosaccharide.

In one embodiment of the foregoing aspect, the prebiotic comprises a sugar selected from the group consisting of arabinose, fructose, fucose, lactose, galactose, glucose, mannose, D-xylose, xylitol, ribose, xylobiose, sucrose, maltose, lactose, lactulose, trehalose, cellobiose, xylooligosaccharide, and combinations thereof.

In one or more embodiments, the present disclosure is directed to dietary supplement or medical food formula that include a combination of at least one human milk oligosaccharide selected from 2′-fucosyllactose (2′-FL) and lacto-N-neotetraose (LNnT), in combination with oligofructose-enriched inulin (OFI), or beta-glucans (BG) or probiotic strains of Bifidobacterium and Lactobacilli strains, where the OFI and BG are present at 3-14 gm and 1-5 gm respectively

Also provided are methods of use of the supplement formulation for improving biomarkers in at least one of the conditions namely Chronic Kidney Disease (CKD), Diabetic Kidney Disease (DKD), dysbiosis and or Gut injury from Chemotherapy, and inflammatory or irritable gut conditions of human subjects.

In one or more embodiments, the dietary supplement or medical food formulas and methods of the present disclosure offer an alternative therapeutic or nutritional intervention option that can contribute to one or more of improved clinical biomarkers in at least one of the conditions namely Chronic Kidney Disease (CKD), Diabetic Kidney Disease (DKD), dysbiosis and or Gut injury from Chemotherapy, and inflammatory or irritable gut conditions in children, youth or adult humans.

### Human Milk Oligosaccharides

In one or more embodiments, the dietary supplements or medical foods described herein include at least one of the human milk oligosaccharides (HMOs)

In one or more embodiments, the human milk oligosaccharides (HMOs), are present in the dietary supplements or medical foods at a concentration effective to improve one or more of clinical biomarkers in at least one of the conditions namely Chronic Kidney Disease (CKD), Diabetic Kidney Disease (DKD), dysbiosis and or Gut injury from Chemotherapy, and inflammatory or irritable gut conditions, including a concentration from about 0.001 mg to 10 gm for solid formulations and for Liquids concentration from 0.0001 mg/mL to about 50 mg/mL, from about 0.001 mg/mL to about 20 mg/mL, from about 0.01 mg/mL to about 10 mg/mL, from about 0.05 mg/mL to about 5 mg/mL, from about 0.5 mg/mL to about 1 mg/mL, or at a concentration of about 0.0001 mg/mL, about 0.0005 mg/mL, about 0.001 mg/mL, about 0.005 mg/mL, about 0.01 mg/mL, about 0.025 mg/mL, about 0.05 mg/mL, about 0.075 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.2 mg/mL, about 1.4 mg/mL, about 1.6 mg/mL, about 1.8 mg/mL, about 2.0 mg/mL, about 5.0 mg/mL, about 7.5 mg/mL, about 10 mg/mL, about 15 mg/mL, about 20 mg/mL, about 25 mg/mL, about 30 mg/mL, about 40 mg/mL, or about 50 mg/mL.

In one or more embodiments, the dietary supplements or medical foods described herein include at least one of the human milk oligosaccharides (HMOs) 2′-FL and LNnT. Research has shown multiple roles for HMOs in improvement of gut flora with increased Bifidobacteria. For example, HMOs have been shown to modulate microbiome, reduce inflammation, including neuroinflammation, to promote the growth of beneficial microorganisms, thereby reducing toxic metabolites such as TMAO. It is applicants' belief that inclusion of at least one of the HMOs 2′-FL and LNnT in the dietary supplement or medical food formula can improve clinical biomarkers, in at least one of the conditions namely Chronic Kidney Disease (CKD), Diabetic Kidney Disease (DKD), dysbiosis and or Gut injury from Chemotherapy, and inflammatory or irritable gut conditions

In one aspect of the present invention, there is provided a nutritional composition comprising at least one fucosylated oligosaccharide and at least one N-acetylated oligosaccharide, for use in the prevention and/or treatment of at least one of the conditions namely chronic kidney disease (CKD), diabetic kidney disease (DKD), dysbiosis and or gut injury from chemotherapy, and inflammatory or irritable gut conditions of human subjects

In one aspect of the present invention, the nutritional composition of the present invention comprises at least one fucosylated oligosaccharide. There can be one or several types of fucosylated oligosaccharide(s). The fucosylated oligosaccharide(s) can indeed be selected from the list comprising 2′-fucosyllactose, 3′fucosyllactose, difucosyllactose, lacto-N-fucopentaose (such as lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose, tri-fuco-para-Lacto-N-hexaose I and any combination thereof.

In some particular embodiments the fucosylated oligosaccharide comprises a 2′-fucosyl-epitope. It can be for example selected from the list comprising 2′-fucosyllactose, difucosyllactose, lacto-N-fucopentaose, lacto-N-fucohexaose, lacto-N-difucohexaose, fucosyllacto-N-hexaose, fucosyllacto-N-neohexaose, difucosyllacto-N-hexaose difuco-lacto-N-neohexaose, difucosyllacto-N-neohexaose, fucosyl-para-Lacto-N-hexaose and any combination thereof.

In one or more embodiments, the nutritional composition according to the invention comprises 2′-fucosyllactose (or 2FL, or 2′FL, or 2-FL or 2′-FL). In a particular embodiment, there is no other type of fucosylated oligosaccharide than 2′-fucosyllactose, i.e. the nutritional composition of the invention comprises only 2′-fucosyllactose as fucosylated oligosaccharide.

In one or more embodiments, the composition of the present invention also comprises at least one the N-acetylated oligosaccharide. There can be one or several types of N-acetylated oligosaccharide. The N-acetylated oligosaccharide(s) can be for example lacto-N-tetraose (LNT), lacto-N-neotetraose (LNnT) or any combination thereof. In some particular embodiments the N-acetylated oligosaccharide is lacto-N-neotetraose (LNnT), para-lacto-N-neohexaose (para-LNnH) or any combination thereof. In some particular embodiments the N-acetylated oligosaccharide is LNnT. In some particular embodiments the N-acetylated oligosaccharide is LNT. In some other particular embodiments, the N-acetylated oligosaccharide is a mixture of LNT and LNnT. In some particular embodiments the composition comprises both LNT and LNnT in a ratio LNT:LNnT between 5:1 and 1:2, or from 2:1 to 1:1, or from 2:1.2 to 2:1.6.

In one or more embodiments, the nutritional composition according to the invention comprises lacto-N-neotetraose (LNnT). In a particular embodiment, there is no other type of N-acetylated oligosaccharide than lacto-N-neotetraose (LNnT), i.e. the nutritional composition of the invention comprises only lacto-N-neotetraose (LNnT) as N-acetylated oligosaccharide.

In a particularly advantageous embodiment of the present invention, the nutritional composition comprises 2′-fucosyllactose (2FL) and lacto-N-neotetraose (LNnT).

In some embodiments the fucosylated oligosaccharide(s): N-acetylated oligosaccharide(s) (e.g. 2FL:LNnT) weight ratio in the nutritional composition of the present invention is from 1:10 to 12:1 such as from 1:7 to 10:1 or from 1:5 to 5:1, or from 2:1 to 5:1 or from 1:3 to 3:1, or from 1:2 to 2:1, or from 1:1 to 3:1, or from 1:5 to 1:0.5.

The fucosylated oligosaccharide(s) and the N-acetylated oligosaccharide(s) present into the nutritional composition of the present invention may be in a total amount of from 0.1 to 10 wt %, such as from 0.5 to 7 wt % or from 1 to 5 wt % of the nutritional composition before reconstitution with water. For reconstituted ready-to-drink formula target is from 0.01 to 1%, more preferably 0.05 to 0.7% or 0.1 to 0.5%.

The nutritional composition of the present invention may for example comprise: fucosylated oligosaccharide(s) in a total amount of 0.2-5 g/L, for example 0.5-4.5 g/L or 1-4 g/L of the composition, or in a total amount of 0.13-3.48 g/100 g, for example 0.34-3.13 g/100 g or 0.69-2.78 g/100 g of composition on a dry weight basis; and/or N-acetylated oligosaccharide(s) in a total amount of 0.05-5 g/L, for example 0.1-2 g/L or 0.1-1 g/L of the composition, or in a total amount of 0.0.03-3.48 g/100 g, for example 0.07-1.4 g/100 g or 0.07-0.7 g/100 g of composition on a dry weight basis.

The nutritional composition according to the present invention may also comprise at least another oligosaccharide(s) (i.e. other than the fucosylated oligosaccharide(s) and N-acetylated oligosaccharide(s) necessarily present in the composition) and/or at least a fiber(s) and/or at least a precursor(s) of human milk oligosaccharide(s). The other oligosaccharide and/or fiber and/or precursor may be selected from the list comprising galacto-oligosaccharides (GOS), fructo-oligosaccharides (FOS), inulin, xylooligosaccharides (XOS), polydextrose, sialylated oligosaccharides, sialic acid, fucose and any combination thereof. They may be in an amount between 0 and 10% by weight of composition.

In a particular embodiment, the composition according to the invention can comprise sialylated oligosaccharide(s). There can be one or several sialylated oligosaccharide(s). The sialylated oligosaccharide(s) can be selected from the group comprising 3′ sialyllactose (3-SL), 6′ sialyllactose (6-SL), and any combination thereof. In some embodiments of the invention the composition comprises 3-SL and 6-SL. In some particular embodiments the ratio between 3′-sialyllactose (3-SL) and 6′-sialyllactose (6-SL) can be in the range between 5:1 and 1:10, or from 3:1 and 1:1, or from 1:1 to 1:10. In some specific embodiments the sialylated oligosaccharide of the composition is 6′ sialyllactose (6-SL).

In particular examples the composition may comprise sialylated oligosaccharide(s) in a total amount of from 0.05 to 5 g/L of, for example from 0.1 to 4 g/L, or from 0.3 to 2 g/L of the composition, or in a total amount of from 0.03 to 3.5 g/100 g, for example from 0.1 to 2 g or from 0.2 to 1 g/100 g of composition on a dry weight basis.

In addition to including at least one of the 2′-FL and the LNnT described above, in certain aspects, the dietary supplements or medicated foods can contain sialylated or fucosylated human milk oligosaccharides (HMOs). Any or all of the HMO(s) used in the dietary supplement or medical foods may be isolated or enriched from milk(s) secreted by mammals including, but not limited to: human, bovine, ovine, porcine, or caprine species. The HMOs may also be produced via microbial fermentation, enzymatic processes, chemical synthesis, or combinations thereof.

Suitable sialylated HMOs for the formulation include at least one sialic acid residue in the oligosaccharide backbone. In certain aspects, the sialylated HMO includes two or more sialic acid residues. Specific non-limiting examples of sialylated HMOs for use in the present disclosure include sialyl oligosaccharides, sialic acid (e.g., free sialic acid, lipid-bound sialic acid, protein-bound sialic acid), lactosialotetraose, 3 ′-Sialyl-3-fucosyllactose, Disialomonofucosyllacto-N-neohexaose, Monofucosylmonosialyllacto-N-octaose (sialyl Lea), Sialyllacto-N-fucohexaose II, Disialyllacto-N-fucopentaose II, Monofucosyldisialyllacto-N-tetraose), sialyl fucosyl oligosaccharides, 2′-Sialyllactose, 2-Sialyllactosamine, 3′-Sialyllactose, 3’-Sialyllactosamine, 6′-Sialyllactose, 6′-Sialyllactosamine, Sialyllacto-N-neotetraose c, Monosialyllacto-N-hexaose, Disialyllacto-N-hexaose I, Monosialyllacto-N-neohexaose I, Monosialyllacto-N-neohexaose II, Disialyllacto-N-neohexaose, Disialyllacto-N-tetraose, Disialyllacto-N-hexaose II, Sialyllacto-N-tetraose a, Disialyllacto-N-hexaose I, Sialyllacto-N-tetraose b, sialyl-lacto-N-tetraose a, sialyl-lacto-N-tetraose b, sialyl-lacto-N-tetraose c, sialyl-fucosyl-lacto-N-tetraose I, sialyl-fucosyl-lacto-N-tetraose II, disialyl-lacto-N-tetraose, and combinations thereof.

Specific non-limiting examples of fucosylated HMOs for the formulation include fucosyl oligosaccharides, Lacto-N-fucopentaose I, Lacto-N-fucopentaose II, 3′-Fucosyllactose, Lacto-N-fucopentaose III, Lacto-N-difucohexaose I, Lactodifucotetraose, monofucosyllacto-N-hexaose II, isomeric fucosylated lacto-N-hexaose (1), isomeric fucosylated lacto-N-hexaose (3), isomeric fucosylated lacto-N-hexaose (2), difucosyl-para-lacto-N-neohexaose, difucosyl-para-lacto-N-hexaose, difucosyllacto-N-hexaosemonofucosyllacto-neoocataose, monofucosyllacto-N-ocataose, difucosyllacto-N-octaose I, difucosyllacto-N-octaose II, difucosyllacto-N-neoocataose II, difucosyllacto-N-neoocataose I, lacto-N-fucopentaose V, lacto-N-decaose, trifucosyllacto-N-neooctaose, trifucosyllacto-N-octaose, trifucosyl-iso-lacto-N-octaose, lacto-N-difuco-hexaose II, and combinations thereof.

In certain aspects, other suitable examples of HMOs for inclusion in the dietary supplements or medical foods include lacto-N-hexaose, para-lacto-N-hexaose, lacto-N-neohexaose, para-lacto-N-neohexaose, lacto-N-neoocataose, para-lacto-N-octanose, iso-lacto-N-octaose, lacto-N-octaose, and combinations thereof.

In one or more embodiments, the oligosaccharide in the composition comprise at least about 0.001 mg to 10 μm and liquids at least about 0.001 mg/mL, at least about 0.01 mg/mL, from about 0.001 mg/mL to about 20 mg/mL, from about 0.01 mg/mL to about 20 mg/mL, from 0.001 mg/mL to about 10 mg/mL, from about 0.01 mg/mL to about 10 mg/mL, from 0.001 mg/mL to about 5 mg/mL, from about 0.01 mg/mL to about 5 mg/mL, from about 0.001 mg/mL to about 1 mg/mL, from about 0.001 mg/mL to about 0.23 mg/mL, or from about 0.01 mg/mL to about 0.23 mg/mL total human milk oligosaccharide in the supplement or medical food. In certain aspects, the amount of specific sialylated human milk oligosaccharide, the fucosylated human milk oligosaccharide, or both (exclusive of the at least one of the 2′-FL and the LNnT) present in the composition will depend on the specific human milk oligosaccharide or human milk oligosaccharides present and the amounts of other components in the supplements or medical food. Synthetic (i.e., non-human or animal sourced) or bacterial sourced HMOs are preferably used in the supplements or medical foods disclosed herein.

### Prebiotic Oligofructose-Enriched Iulin (OFS)

The dietary supplement or medical foods described herein include the OFS sourced from chicory root. Prebiotics are nondigestible (oligo) saccharides, defined as “selectively fermented ingredients that allow specific changes, both in the composition and/or activity of the gastrointestinal microflora that confers benefits upon host well-being and health.”

Substances are considered prebiotics when they meet the following criteria: 1) be neither hydrolyzed nor absorbed in the upper part of the gastrointestinal tract; 2) be selectively fermented by 1 or a limited number of potentially beneficial bacteria in the intestine; and 3) be able to alter the colonic microflora toward a healthier composition.

Inulin is a food ingredient consisting of a unique combination of inulin fractions with selected chain lengths. The shorter chain inulin (oligofructose) is combined with longer chain inulin either in equal amounts or at a ratio as required for clinical efficacy in different disease conditions. Inulin consists of Oligo- and polysaccharides composed of fructose units and every fructose chain is terminated by a glucose unit. The number of fructose and glucose units in inulin (degree of polymerization=DP) ranges between 2 and 60. Oligofructose is a degradation product of inulin and consists of fructose polymers with a blend of higher and lower degree of polymerization for desired physiological effects. Research has shown that inulin fructans are selectively fermented by residential microbiota into short chain fatty acids (SCFAs) and lactate. It is applicants' belief that Inulin fructans in combination of at least one of the HMOs 2′-FL and LNnT along with betaglucans and probiotic strains (Bifidobacter and Lactobacelli) in the dietary supplement or medical food formula can improve clinical biomarkers, in at least one of the conditions namely Chronic Kidney Disease (CKD), Diabetic Kidney Disease (DKD), dysbiosis and or Gut injury from Chemotherapy, and inflammatory or irritable gut conditions Beta-Glucan

The oat beta-glucans described herein are known to promote a saccharolytic shift in microbial metabolism. Our supplement with combination of HMOs generates additional health properties of beta-glucans. In particular, they induce a modulation of the gut microbiota taxonomic composition and metabolism, leading to an increase of SCFA levels, thereby reducing TMAO and pCS blood levels. In the context of CKD, where the excretory function of kidney declines, Beta Glucan supplementation can effectively reduce the pCS (uremic toxins) and reduce accumulation in blood in a several fold proportion.

It is applicants' belief that betaglucans in combination with at least one of the HMOs 2′-FL and LNnT along with Inulin fructans and probiotic strains (Bifidobacter and Lactobacelli) in the dietary supplement or medical food formula improve clinical biomarkers in one or more clinical conditions selected from Chronic Kidney Disease (CKD), Diabetic Kidney Disease (DKD), dysbiosis and or Gut injury from Chemotherapy, and inflammatory or irritable gut conditions

In certain aspects, the supplement or medical foods includes at least about 0.001 gm to 5 gm of one or both of Inulin and beta glucan. If used in liquid formulations, the supplement include Inulin fructans (OFS) in a concentration of at least 60 mg/L, including at least 70 mg/L, including at least 80 mg/L, including at least 90 mg/L, including at least 100 mg/L, including at least 150 mg/L, including at least 200 mg/L and including from 60 mg/L to about 1000 mg/L, and including from about 100 mg/L to about 500 mg/L. In certain liquid formulations the betaglucan in a concentration of at least 110 mg/L, including at least 120 mg/L, including at least 130 mg/L, including at least 140 mg/L, including at least 150 mg/L, including at least 200 mg/L, and including from 110 mg/L to about 1000 mg/L, and also including from about 110 mg/L to about 500 mg/L. In addition inulin and betaglucan, in certain aspects, the supplement formula includes one or more additional prebiotic fibers. Such additional prebiotic fibers include, but are not limited to, partially hydrolyzed guar gum, guargum, guar fiber, wheat or malt dextrins. The prebiotic fibers may be derived from plant sources, marine plankton, fungal or synthetic.

### Probiotics

The nutritional composition of the present invention further comprises at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM I-2116, Lactobacillus johnsonii CNCM I-1225, Lactobacillus plantarum UALp-05, Streptococcus salivarius DSM 13084, Bifidobacterium lactis CNCM 1-3446 sold inter alia by the Christian Hansen company of Denmark under the trademark Bb 12, Bifidobacterium longum ATCC BAA-999, Bifidobacterium breve, Bifidobacter Bifidum, Bifidobacterium infantis sold by Procter & Gamble Co. under the trademark Bifantis and Bifidobacterium breve sold by Institut Rosell (Lallemand) under the trademark R0070.

The nutritional composition according to the invention may contain from 10³ to 10¹² cfu of probiotic strain, more preferably between 10⁷ and 10¹² cfu such as between 10⁸ and 10¹⁰ cfu of probiotic strain per g of composition on a dry weight basis.

In one embodiment the probiotics are viable. In another embodiment the probiotics are non-replicating or inactivated. There may be both viable probiotics and inactivated probiotics in some other embodiments.

In one or more embodiments, the probiotics present in the dietary supplements as an ingredient or additive can be prokaryotes, eukaryotes, or archaebacteria. Examples of suitable probiotics include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. In preferred embodiments, the probiotics comprise at least one of any suitable strain or subspecies of Enterococcus, Streptococcus, Lactobacillus, Lactococcus, Bifidobacterium, or Saccharomyces. Enterococcus species include, without limitation, Enterococcus facecium, specifically Enterococcus faecium strain SF68 (NCIMB 10415), as well as other Enterococci Streptococcus species including, without limitation, Streptococcus faecium, Streptococcus thermophilus, and Streptococcus salivarus. Lactobacillus species include, without limitation, Lactobacillus acidophilus, Lactobacillus acidophilus NCC2628 (CNCM 1-2453), Lactobacillus acidophilus NCC2766, Lactobacillus acidophilus NCC2775, Lactobacillus brevis, Lactobacillus bulgaricus, Lactobacillus casei, Lactobacillus casei Imunitas, Lactobacillus casei Shirota, Lactobacillus cellobiosus, Lactobacillus crispatus, Lactobacillus curvatus, Lactobacillus delbrueckii, Lactobacillus fermentum, Lactobacillus gasseri, Lactobacillus helveticus, Lactobacillus johnsonii, Lactobacillus johnsonii NCC2774, Lactobacillus johnsonii NCC2767, Lactobacillus johnsonii NCC2822, Lactobacillus plantarum, Lactobacillus reuteri, Lactobacillus johnsonii LA1, Lactobacillus plantarum, Lactobacillus reuteri, Lactobacillus reuteri NCC2823, Lactobacillus reuteri NCC2581 (CNCM 1-2448), Lactobacillus reuteri NCC2592 (CNCM 1-2450), Lactobacillus reuteri NCC2603 (CNCM I-2451), Lactobacillus reuteri NCC2613 (CNCM 1-2452), Lactobacillus rhamnosus Lactobacillus rhamnosus NCC2583 (CNCM 1-2449), Lactobacillus rhamnosus GG (ATCC 53103; Lactobacillus rhamnosus or Lactobacillus casei subspecies rhamnosus), Lactobacillus salivarius, Lactobacillus salivarius NCC2586, Lactobacillus paracasei ST11. Lactococcus species include, without limitation, Lactococcus lactis and Lactococcus plantarum. Bifidobacterium species include, without limitation, Bifidobacterium adolescentis, Bifidobacterium bifidum, Bifidobacterium animalis, Bifidobacterium thermophilum, Bifidobacterium breve, Bifidobacterium longum, Bifidobacterium pseudolongum, Bifidobacterium infantis, Bifidobacterium lactis, Bifidobacterium lactis Bb-12, B. angulatum, B. asteroides, B. catenulatum, B. choerinum, B. coryneforme, B. caniculi, B. dentium, B. globosum, B. indicum, B. magnum, B. minimum, B. pseudocatenulatum, B. pullorum, B. subtile, B. suis. Saccharomyces species include, without limitation, Saccharomyces boulardii (cerevisiae). In preferred embodiments, the dietary supplements comprise Enterococcus faecium (SF68).

In one or more embodiments, the dietary supplements comprise probiotics in an amount suitable for enhancing the palatability of the probiotics and compositions containing the probiotics, enhancing the immune system to augment the beneficial effects of the probiotics, and/or extending the life of the probiotics. Generally, the dietary supplements comprise from about 1% to about 90% probiotics, preferably from about 1% to about 70% probiotics, most preferably from about 1% to about 50% probiotics.

Generally, probiotics are administered to the subject in amounts of from about one to about fifty billion colony forming units (CFUs) per day for the healthy maintenance of intestinal microflora. The dietary supplements can be formulated to contain probiotics in the range of about 10⁵ to about 10¹⁰ colony forming units (CFU) per gram of the supplement, although higher concentrations of probiotics can be supplied.

In an embodiment, the probiotic strain is about 100 billion CFU, 50 billion CFU, 25 billion CFU, 20 billion CFU, 10 billion CFU, 5 billion CFU, 1 billion CFU, 0.5 billion CFU or less.

The dietary supplements of the invention can comprise additional substances such as minerals, vitamins, salts, proteins, amino acids, fibers, condiments, colorants, and preservatives. Non-limiting examples of minerals include calcium, phosphorous, potassium, sodium, iron, chloride, boron, copper, zinc, magnesium, manganese, iodine, selenium and the like, and various salts thereof. Non-limiting examples of vitamins include vitamin A, various B vitamins, e.g., niacin, pantothenic acid, folic acid, biotin, vitamin C, D, E and vitamin K. The dietary supplements may also comprise carotenoids such as alpha-carotene, lycopene, lutein, zeaxanthin, Astaxanthin and beta-cryptoxanthin. Additional functional ingredients may also be included, for example, Curcumin, glucosamine, chondroitin, collagen, avocatin B, Pycnogenol, ashwagandha, fruit and or berry extracts (such as cranberry, pomegranate, elderberry, chokeberry) amino acids, and the like. One particularly preferred amino acid is taurine.

In various embodiments, the dietary supplements of the invention may further comprise from about 15% to about 60% crude protein. Preferably, the compositions comprise about 40% to about 55% crude protein. The crude protein material may comprise vegetable proteins such as soybean, corn, rice, cottonseed, and peanut, or animal proteins such as casein, albumin, and meat protein. Non-limiting examples of meat protein useful herein include pork, lamb, equine, poultry, fish, and mixtures thereof.

The dietary supplements may further comprise from about 5% to about 40% fat. Preferably, the dietary supplements comprise about 15% to about 22% fat. The dietary supplements may further comprise a source of carbohydrate. The dietary supplements may comprise from about 15% to about 60% carbohydrate. Non-limiting examples of such carbohydrates include grains or cereals such as rice, corn, sorghum, alfalfa, barley, soybeans, canola, oats, wheat, and mixtures thereof. The dietary supplements may also optionally comprise other materials such as dried whey and other dairy by-products.

The dietary supplements may also further comprise at least one fiber source. The dietary supplement may comprise from about 0.5% to about 5% fiber. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, mannanoligofructose, soy fiber, fiber from lupins, arabinogalactan, galactooligosaccharide, arabinoxylan, or mixtures thereof. The fiber source can be a fermentable fiber, as are many of those listed above. Fermentable fiber has previously been described to provide a benefit to the immune system of companion animals. Fermentable fiber or other compositions known to those of skill in the art which provide a prebiotic composition that could enhance the growth of probiotics within the intestine may also be incorporated into the composition to aid in the enhancement of the benefits provided by the present invention to the immune system gastrointestinal system, and general health of an animal.

To enhance the length of time the dietary supplements can be stored, the dietary supplements preferably have a total moisture content between about 2% and about 10% by weight of the supplement. Preferably, the total moisture content is less than 5% by weight of the supplement. Similarly, the dietary supplement preferably has a water activity in the range of 0.20 to 0.6. Preferably, the water activity is less than 0.55.

The dietary supplements can be specially formulated for humans as well as particular animals such as dogs or cats. Similarly, the dietary supplements may be specially formulated for young, adult, or senior animals. In general, specialized formulations comprise ingredients that meet the energy and nutritional requirements appropriate for particular animals and for particular animals at different stages of development or age, or with specific nutrient requirements related to a disease state.

In one embodiment, the probiotics are microencapsulated. The probiotics can be microencapsulated within a biocompatible microcapsule to enhance or sustain the viability of the probiotics, particularly during storage. The microcapsule acts as a barrier to protect the probiotics from harmful environmental conditions such as temperature fluctuations, oxygen, moisture, and light. Microcapsules comprise an active agent, e.g., a probiotic core surrounded by a biocompatible shell or coating. Suitable materials for preparation of biocompatible microcapsules and methods for encapsulation are known in the art, see for example, U.S. Pat. No. 6,974,592 to Yan, U.S. Pat. No. 6,969,530 to Curtis et al., U.S. Pat. No. 6,887,493 to Shefer et al. and U.S. Pat. No. 6,303,355 to Opara, and U.S. patent application Ser. No. 10/507,359 to Ubbink et al., and Ser. No. 10/656,386 to Simmons et al., the contents of each are incorporated by reference herein. The microcapsules can be prepared for controlled release of the probiotics after administration to an animal. In preferred embodiments, the microcapsules of the invention range from about 0.1 mm to about 1.0 mm in diameter. In one embodiment, the probiotics are microencapsulated with a biopolymer matrix coated with shellac.

The skilled artisan can determine the appropriate amount of probiotics to be added to a given dietary supplement formulation. Such factors that may be taken into account include the average consumption of specific types of compositions by different animals, whether the animal that is intended to ingest the dietary supplements has any particular health, wellness, or nutritional requirements, or suffers from a particular disease or disorder, the age, sex, size, or breed of the animal, and the manufacturing conditions under which the dietary supplement composition is prepared. The concentrations of probiotics to be added to the supplement can be calculated on the basis of the energy and nutrient requirements of the animal.

The methods of use of the present disclosure include the oral administration of the supplements that include Inulin fructans, Beta Glucans, Probiotics (Bifidobacter and Lactobacelli), and at least one of 2′-FL and LNnT to improve clinical biomarkers in at least one of the conditions namely Chronic Kidney Disease (CKD), Diabetic Kidney Disease (DKD), dysbiosis and or Gut injury from Chemotherapy, and inflammatory or irritable gut syndrome conditions.

The supplement or medical food as described herein can be administered to individuals with insufficient renal excretion such as CKD, damage to gut epithelium from chemicals such as in cancer chemotherapy or inflammation of the gut from dysbiosis and conditions that can impair/reduce normal gut flora.

The individual desirably consumes at least one serving of the supplement or medical food formulation daily, and in some embodiments, may consume two, three, or even more servings per day. Each serving is desirably administered as a single, undivided dose, although the serving may also be divided into two or more partial or divided servings to be taken at two or more times during the day. The methods of the present disclosure include continuous day after day administration, as well as periodic or limited administration, although continuous day after day administration is generally desirable. The methods of the present disclosure are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least about 18-24 months, desirably as a long term, continuous, daily medical food or supplement.

In another aspect, the present invention provides a method for administering a probiotic to an animal comprising administering to the animal a food composition comprising one or more ingredients suitable for consumption by an animal and a dietary supplement of the present invention.

In another aspect, the present invention provides a method for manufacturing a food composition containing probiotics comprising admixing one or more ingredients suitable for consumption by an animal and a dietary supplement of the present invention or applying a dietary supplement of the present invention onto the food composition. In a further aspect, the present invention provides the food compositions manufactured using this method.

In another aspect, the present invention provides a method for promoting the health or wellness in an animal comprising administering to an animal a health or wellness promoting amount of the dietary supplement of the present invention. In one embodiment, the dietary supplement is administered in conjunction with an anti-diarrhea agent.

In another aspect, the present invention provides a method for promoting gastrointestinal health in an animal comprising administering to an animal a gastrointestinal health promoting amount of the dietary supplement of the present invention. In one embodiment, the dietary supplement is administered in conjunction with an anti-diarrhea agent.

For these methods, the supplement can be administered using any suitable method but is preferably administered by admixing the supplement with or applying the supplement onto a food composition, preferably a nutritionally complete food composition.

To promote health and wellness or to promote gastrointestinal health, the probiotics are administered to the animal via the dietary supplement of the present invention in amounts of from about one to about fifty billion colony forming units (CFUs) per day

### Additional Agents

In some examples, the composition can further include d-ribose, folic acid, malic acid, vitamin B6, vitamin B12, vitamin D3, magnesium oxide, calcium, inulin, chicory root extract, cherry extract, or a combination thereof. In some examples, the composition can further include a pharmaceutically acceptable carrier. In one example, the composition can further include coatings, isotonic agents, absorption delaying agents, binders, adhesives, lubricants, disintegrants, coloring agents, flavoring agents, sweetening agents, absorbents, detergents, emulsifying agents, antioxidants, vitamins, minerals, proteins, fats, carbohydrates, or a combination thereof. In one example, the composition can further include a sweetener, a preservative, a flavoring, or a combination thereof. In some examples, the formulation can include a polymers for sustained release of a given compound.

In another example, the formulation can include emulsifiers. In one example, the emulsifier can add stability to the final product. Examples of suitable emulsifiers include, but are not limited to, lecithin (e.g., from egg or soy), or mono- and di-glycerides. Other emulsifiers are readily apparent to the skilled artisan and selection of suitable emulsifier(s) will depend, in part, upon the formulation and final product.

In yet another example, the formulation can include a preservative. In one example, the preservatives such as potassium sorbate, sodium sorbate, potassium benzoate, sodium benzoate, or calcium disodium EDTA are used.

In a further example, the nutritional supplement can contain natural or artificial sweeteners, e.g., glucose, sucrose, fructose, saccharides, cyclamates, aspartame, sucralose, aspartame, acesulfame K, or sorbitol.

In one example, the composition can be in the form of an oral dosage formulation. In another example, the oral dosage formulation can be a capsule, a tablet, a soft gel, a lozenge, a sachet, stick pack, a powder, a beverage, a syrup, a suspension, or a food. In another example, the compositions can be formulated into a food or drink, and provided, for example, as a snack bar, a cereal, a drink, a gum, or in any other easily ingested form. In one example, the composition can be incorporated into a liquid beverage such as water, milk, juice, or soda. In another example, the composition can be formulated into a nutritional beverage. The nutritional beverage can be in a premixed formulation or can be a powdered mix in that can be added to a beverage. In another example, the powder mix in can be in the form of granules. In one example, the composition can be dried and made readily soluble in water.

In yet another example, the oral dosage form can be in a solution or a suspension in an aqueous liquid or non-aqueous liquid, such as ethanol, glycerol, vegetable oil, salt solutions, or hydroxymethyl cellulose; or in the form of an oil-in-water emulsion or a water-in-oil emulsion, or a combination thereof. In examples where the oral dosage form includes oils, the oils can be edible oils, such as e.g. cottonseed oil, sesame oil, coconut oil, or peanut oil. In some examples, the composition can include suitable dispersing or suspending agents for aqueous suspensions include synthetic or natural gums such as tragacanth, alginate, gum arabic, dextran, sodium carboxymethylcellulose, gelatin, methylcellulose, and polyvinylpyrrolidone.

In another example, the composition can be formulated into a food product. In one example, the food product can be a pudding, confections, (i.e., candy), ice cream, frozen confections and novelties, or non-baked extruded food products such as bars. In one example, the composition can be a powder that is added to non-baked goods. For, example a nutritional bar can be manufactured by adding the powder to the dry ingredients and then incorporating the dry and wet. The wet and dry ingredients can be mixed until the dough phase is reached. The dough can then be put into an extruder and extruded; the extruded dough can be cut into appropriate lengths; and the product can be cooled.

Flavors, coloring agents, spices, nuts, and the like can be incorporated into the product. Flavorings can be in the form of flavored extracts, volatile oils, chocolate flavorings (e.g., non-caffeinated cocoa or chocolate, chocolate substitutes such as carob), peanut butter flavoring, cookie crumbs, crisp rice, vanilla, or any commercially available flavoring. Flavorings can be protected with mixed tocopherols. Examples of useful flavorings include but are not limited to pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, cherry oil, walnut oil, cinnamon oil, clove oil, or peppermint oil; peanut butter, chocolate flavoring, vanilla cookie crumb, butterscotch, or toffee. In one example, the nutritional supplement contains berry or other fruit flavor. The food compositions may further be coated, for example with a yogurt coating if it is as a bar.

In one example, the formulation can be in the form of a cream or lotion for topical application. In another example the active ingredient can be in the form of a bolus, electuary, or paste. In yet another example, the composition can be formulated as a depot preparation. In one example, the depot can be for implantation (e.g. subcutaneously, intra-abdominally, or intramuscularly) or intramuscular injection. In one example, the formulation can be formulated as an ion exchange resin.

Table 1 below provides a formulation according to present invention.

TABLE 1
Formulation according to an embodiment
of present invention.

| Synbiotic | Per Stick Pack | |
|---|---|---|
| Inulin | 5.5 | gm |
| Suncran | 2 | gm |
| 2FL (HMO) | 1.5 | gm |
| Oat Beta Glucan | 0.75 | gm |
| Astaxanthin | 0.1 | gm |
| Probiotics | 0.55 | gm |
| (Bifidobacter Bifidum | | |
| Bifidobacter Longum | | |
| Lactobacillus Plantarum; or | | |
| Lactobacillus Rhamnosus) | | |

The above disclosure generally describes the present invention. A more complete understanding can be obtained by referring to the following examples. These examples are described solely for purposes of illustration and are not intended to limit the scope of the invention. Although specific terms have been employed herein, such terms are intended for descriptive use and not for purposes of limitation.

### Example 1: Sustained Remission in a Pediatric Case of Crohns with Use of the Synbiotic Adjunt to the Partial Enteral Nutrition

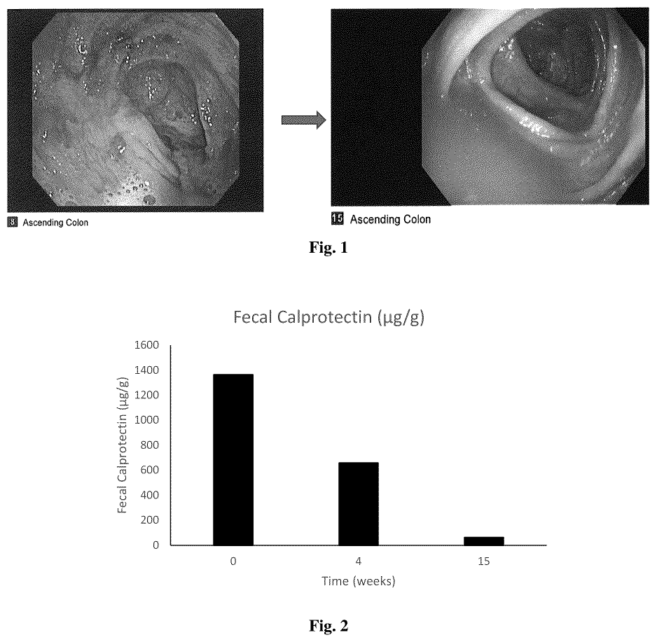

Background and Case History: 11-year-old Asian male with abdominal pain and weight loss, referred to pediatric gastroenterology clinic by hematology for refractory iron deficiency anemia. He had extensive work up including labs, stool studies, upper endoscopy, video capsule (VCE) and lower endoscopies. Work up for infections and celiac disease were negative. His stool elastase was elevated to 1375 μg/g (normal <50 μg/g), Iron studies showed-total iron at 20 mcg/dL (normal 50-120 mcg/dL), Ferritin at 53.4 ng/ml (normal 10-322 ng/ml), Iron saturation at 6% (normal 20-55%). Vitamin D level was at 12.7 ng/ml (normal 32-100 ng/ml). VCE showed discreet linear and serpiginous ulcers with surrounding erythema in duodenum and proximal jejunum. Colonoscopy was grossly normal but biopsies showed multiple granulomas in terminal ileum and entire colon suggesting Crohn's disease (Inflammatory phenotype) (FIG. 1). The decision was made for him to be started on Humira infusions. He was on liquid diet as he could not tolerate solids due to pain.

Treatment Approach: Anti-TNF (Tumour Necrosis Factor) medications were proposed but lacked consent from family. After discussion with family he was started on Exclusive Enteral Nutrition (EEN) with polymeric formula and went into clinical and biochemical remission within eight weeks. After remission we did slow introduction to solid foods as an attempt for partial wean from EEN, that caused a flare with calprotectin going to 1375 μg/g (0-120 μg/g normal) increased pain episodes. At that time patient was started on proprietary Synbiotic formulation (combination of human milk oligosaccharide (HMO), inulin, astaxanthin and probiotics blend with strains of Lactobacilli and Bifidobacterium) and no other treatment. After 4 weeks with Synbiotic, fecal calprotectin levels decreased to 665 μg/g which further dropped to 110 μg/g by week 12. Patient continued tolerance for solid foods with improved appetite and no pain symptoms. We continued slow relaxation of diet—80% polymeric formula and 20% specific foods rich in protein and fiber with eventual goal of maintaining remission at 50% enteral polymeric formula and 50% diet.

Results: One-year from initial diagnosis and 15 weeks of Synbiotic supplement patient had complete evaluation showing resolution of anemia, normal iron studies (total iron 125 and iron saturation 23), calprotectin at 65 μg/g (FIG. 2), mucosal healing on scopes and biopsies with no granulomas or inflammation suggesting deep histological remission (FIG. 1).

Conclusions: Published literature for pediatric Crohn's disease showed remission with EEN, but very limited data on maintenance of remission on partial enteral nutrition (PEN). As it would be challenging for patients to continue to be on EEN and with increasing knowledge on the importance of the gut microbiome in IBD, we aimed to nourish and restore the gut-biome using Synbiotic as a novel approach. Here with the use of Synbiotic along with PEN we show sustained deep remission in 15 weeks. Considering the pivotal role of the microbiota in IBD, new therapeutic approaches such as HMO based Synbiotics with potent antioxidants might be the missing piece of the puzzle. HMO based Synbiotic formulation foster interspecies interactions, cross feeding, and generation of metabolites (SCFAs), providing a holistic and superior approach for remission in IBD.

1. A synbiotic formulation, comprises: a) human milk oligosaccharide (HMO) comprising 2′-fucosyllactose (2′-FL) and/or lacto-N-neotetraose (LNnT), b) an anti-oxidant comprising astaxanthin and c) a probiotic strain of microorganism comprising Bifidobacter species and/or Lactobacillus species;
Wherein the probiotic strain is about 20 billion CFU, HMO is about 0.5-10 g and antioxidant of about 0.01-2 g in a total weight of the symbiotic formulation.

2. The synbiotic formulation of claim 1 comprising between about 1 g/mL and about 10 g/mL of at least one human milk oligosaccharide.

3. The synbiotic formulation of claim 2, comprising between about 1 g/mL to about 2 g/mL of at least one human milk oligosaccharide.

4. The synbiotic formulation of claim 1, wherein HMO comprises 2′FL.

5. The synbiotic formulation of claim 1, further comprises inulin and betaglucan.

6. (canceled)

7. The synbiotic formulation of claim 5, wherein the inulin is present between about 3 g/mL to about 7 g/mL and betaglucan is present between about 0.4 g/mL to about 1 g/mL.

8. The synbiotic formulation of claim 1, further comprises fibers from guar bean and cranberry juice powder.

9. The synbiotic formulation of claim 8, wherein suncran is present between 1 g/mL to about 4 g/mL.

10. The synbiotic formulation of claim 1, comprises Bifidobacterium species comprises B. bifidum and B. longum and Lactobacillus species comprises L. plantarum and L. rhamnosus.

11. The synbiotic formulation of claim 1, is configured to improve a clinical biomarker in a disease comprising Crohn's disease, Chronic Kidney Disease (CKD), Diabetic Kidney Disease (DKD), Dysbiosis, Gut injury from Chemotherapy.

12. A method to treat Crohn's disease comprises:
a) taking a formulation comprising a) human milk oligosaccharide (HMO) comprising 2′-fucosyllactose (2′-FL) and/or lacto-N-neotetraose (LNnT), b) an anti-oxidant comprising astaxanthin and c) a probiotic strain of microorganism comprising Bifidobacter species and/or Lactobacillus species; and
b) administering the formulation in the subject on a regular interval basis.

13. The method of claim 11, wherein the formulation comprises between about 1 g/mL and about 10 g/mL of at least one human milk oligosaccharide.

14. The method of claim 12 wherein the synbiotic formulation comprises from about 1 g/mL to about 2 g/mL of at least one human milk oligosaccharide.

15. The method of claim 11, wherein the synbiotic formulation further comprises inulin and betaglucan.

16. The method of claim 15, wherein inulin is present between about 3 g/mL to about 7 g/mL and betaglucan is present between about 0.4 g/mL to about 1 g/mL.

17. The method of claim 12, wherein administrating of the synbiotic formulation is done at least for 8 weeks for improving a clinical biomarker.

18. The method of claim 17, wherein the clinical biomarker comprises anemia and/or calprotectin.

19. The method of claim 12, wherein formulation induced formation of mucosal tissue in an intestine.

20. A method of a formulation comprising:
a) obtaining a live strain of Bifidobacterium and/or Lactobacilli by freeze-drying; and
b) mixing the live strain with antioxidant comprising astaxanthin and a human milk comprising 2′-FL and/or LNnT;
wherein the formulation is configured to treat crohn's disease.

21. A method of a formulation of claim 21, wherein the formulation in the form of powder, solution, paste, chewable gummy, decoction, tablet, bar.

22. (canceled)
